(19) United States
(12) Patent Application Publication
(10) Pub. No.: US 2010/0119732 A1
(43) Pub. Date: May 13, 2010

(54) HYDROGENATED AMORPHOUS CARBON COATING

(75) Inventors: M. Creatore, Veldhoven (NL); Roland Groenen, Kortrijk (BE); S. V. Singh, Eindhoven (NL); M.C.M. Van De Sanden, Tilburg (NL); Kris Van Hege, Herzele (BE)

Correspondence Address: FOLEY AND LARDNER LLP;SUITE 500, 3000 K STREET NW, WASHINGTON, DC, 20007, US

(73) Assignee: NV Bekaert SA

(21) Appl. No.: 12/524,793

(22) PCT Filed: Feb. 5, 2008
(86) PCT No.: PCT/EP2008/051388

§ 371 Date: Jul. 28, 2009

(30) Foreign Application Priority Data
Feb. 6, 2007 (EP) 07101781.8

(51) Int. Cl. H05H 1/24; C07C 15/00

(52) U.S. Cl. 427/577; 585/24

(57) ABSTRACT

The invention relates to a hydrogenated amorphous carbon coating characterized by a substantial absence of sp^{x} hybridized CHₓ endgroups with x equal to 1, 2 and 3. The invention further relates to a method to deposit such a hydrogenated amorphous carbon coating on a substrate.

### FIELD OF THE INVENTION

The invention relates to a hydrogenated amorphous carbon coating. The invention further relates to a method to deposit such a hydrogenated amorphous carbon coating.

### BACKGROUND OF THE INVENTION

Hydrogenated amorphous carbon has been demonstrated to be a material with a wide range of electronic, optical and tribological properties.

The properties of hydrogenated amorphous carbon are mainly determined by the ratio of sp³ and sp² hybridized carbon and by the hydrogen content.

As the ratio of sp³ and sp² hybridized carbon and the hydrogen content can vary within a broad range, a great variety of the properties of hydrogenated amorphous carbon is possible.

Hydrogenated amorphous carbon coatings can be deposited by a number of different techniques as for example by ion beam deposition, plasma sputtering, laser ablation and most importantly by chemical vapor deposition (CVD), more particularly by plasma enchanced chemical vapor deposition (PECVD).

Furthermore, hydrogenated amorphous carbon coatings can be deposited by techniques using a direct plasma or by techniques using a remote plasma.

In direct plasmas high energy ions are involved. The presence of ion bombardment leads to a growth process that is physical in nature and in which the chemical identity of the growth precursors is suppressed.

In remote plasmas the induced self-bias on the surface can be intrinsically lower than that induced in direct plasmas.

When the electron temperature is low the plasma behavior is ruled by the heavy particle kinetics, i.e. the generation of precursors for growth is dominated by the chemistry and not by physical processes such as electron impact dissociation or ionisation. The chemistry could consequently be more selective leading to the dominant production of one specific precursor for growth.

The technique of using an Expanding Thermal Plasma (ETP) emerged as a promising technique to deposit hydrogenated amorphous carbon coatings at relatively high deposition rates.

Deposition of amorphous hydrogenated carbon coatings by the expanding thermal plasma technique is known from “Optical and mechanical properties of plasma-beam-deposited amorphous hydrogenated carbon, J. Appl. Phys. 80 (10), 5986-5995, 1996”.

Such an amorphous hydrogenated carbon coating is deposited employing a ratio of carrier gas flow to carbon-containing precursor gas flow F higher than 10.

The Fourier Transform InfraRed (FTIR) transmission spectrum of a such an amorphous hydrogenated carbon coating is illustrated in FIG. 3.

A drawback of this coating is the limited hardness.

Fast deposition of high quality hydrogenated amorphous carbon coatings remains therefore an important issue.

### SUMMARY OF THE INVENTION

It is an object of the present invention to provide a hydrogenated amorphous carbon coating characterized by a substantial absence of the sp^{x} hybridized CHₓ endgroups, whereby x is equal to 1, 2 and 3.

It is another object to provide a hydrogenated amorphous carbon coating characterized by a low percentage of sp³ hybridized carbon but nevertheless having a high hardness.

It is a further object to provide a method to deposit a hydrogenated amorphous carbon coating at a high deposition rate.

According to a first aspect of the present invention a hydrogenated amorphous carbon coating characterized by a substantial absence of the sp^{x} hybridized CHₓ endgroups (with x equal to 1, 2 and 3) is provided. The hydrogenated amorphous carbon coating according to the present invention is thus characterized by a substantial absence of the sp¹ hybridized CH₁ endgroups, by a substantial absence of the sp² hybridized CH₂ endgroups and by a substantial absence of the sp³ hybridized CH₃ endgroups.

The sp^{x} hybridized CHₓ groups with x=2 and x=3 serve as endgroups in the bond chain.

As the hydrogenated amorphous carbon coating according to the present invention is characterized by a substantial absence of sp^{x} hybridized CHₓ endgroup a strong interconnected network of C—C bonds is present.

High amounts of sp^{x} hybridized CHₓ endgroups on the other hand result in soft materials.

The substantial absence of the sp^{x} hybridized CHₓ endgroups of a hydrogenated amorphous carbon coating according to the present invention is thus a considerable advantage as this results in hydrogenated amorphous carbon coatings having a high hardness.

The substantial absence of the sp^{x} hybridized CHₓ endgroups is clear from a Fourier Transform InfraRed (FTIR) transmission spectrum. A FTIR transmission spectrum of an amorphous hydrogenated carbon coating according to the present invention shows two peaks separated by a peak valley in the wavenumber range between 2800 and 3400 cm⁻¹, whereas the FTIR transmission spectrum of an amorphous hydrogenated carbon coatings known in the art (J. Appl. Phys. 80 (10), 5986-5995, 1996) shows one broad peak in the wavenumber range between 2800 and 3400 cm⁻¹.

The difference between the two FTIR transmission spectra is clear by determining the first derivative in the wavenumber range between 2800 and 3400 of the FTIR transmission spectra of an amorphous hydrogenated carbon coating according to the present invention and of an amorphous hydrogenated carbon coating known in the art.

The first derivative of a FTIR transmission spectrum in the wavenumber range between 2850 and 3050 cm⁻¹ of a hydrogenated amorphous carbon coating according to the present invention has at least three zero axis crossings.

In a preferred embodiment the first derivative of a FTIR transmission spectrum of a hydrogenated amorphous carbon coating has three zero axis crossings.

A zero axis crossing of the first derivative of a FTIR transmission spectrum is defined as a point corresponding with the intersection of the first derivative with the axis Y=0.

For the purpose of this invention only intersections of the first derivative of a FTIR transmission spectrum with the axis Y=0 in the wavenumber range from 2850 cm⁻¹ to 3050 cm⁻¹ are considered as zero axis crossings. This means that intersections of the first derivative of a FTIR transmission spectrum with the axis Y=0 in the wavenumber range below 2850 cm⁻¹ and in the wavenumber range above 3050 cm⁻¹ are not considered as zero axis crossings.

One zero axis crossing of the at least three zero axis crossings of the first derivative of the FTIR transmission spectrum of an amorphous hydrogenated carbon coating according to the present invention is corresponding with the maximum absolute intensity of the first peak in the FTIR transmission spectrum. A second zero axis crossing is corresponding with the minimum of the absolute intensity in the FTIR transmission spectrum, i.e. with the peak valley A third zero axis crossing is corresponding with the maximum absolute intensity of the second peak in the FTIR transmission spectrum.

On the contrary the first derivative of the FTIR transmission spectrum in the wavenumber range between 2850 and 3050 cm⁻¹ of an amorphous hydrogenated carbon coating known in the art has at least one zero axis crossing.

In a preferred embodiment the first derivative of the FTIR transmission spectrum of an amorphous hydrogenated carbon coating known in the art has one zero axis crossing.

The at least one zero axis crossing of the first derivative of the FTIR transmission spectrum of an amorphous hydrogenated carbon coating according to the prior art is corresponding with the maximum absolute intensity of the peak in the FTIR transmission spectrum.

In any case, the number of zero axis crossings of a FTIR transmission spectrum of a hydrogenated amorphous carbon coating according to the present invention is higher than the number of zero axis crossings of a FTIR transmission spectrum of a hydrogenated amorphous carbon coating known in the the prior art.

As mentioned above, the hydrogenated amorphous carbon coating according to the present invention is in particular characterized by a substantial absence of the sp¹ hybridized CH endgroups, by a substantial absence of sp² hybridized CH₂ endgroups and by a substantial absence of the sp³ hybridized CH₃ endgroups. From the FTIR transmission spectra it is clear that the substantial absence of the sp^{x} hybridized CHₓ endgroups (with x equal to 1, 2 and 3) is the result of the substantial absence of the corresponding stretching vibrations. The substantial absence of a specific sp^{x} hybridized CHₓ endgroup is clear by a substantial absence of the corresponding sp^{x} CHₓ stretching vibration or vibrations in a FTIR transmission spectrum.

The substantial absence of sp¹ hybridized CH endgroups is shown
by a substantial absence of the sp¹ CH stretching vibration at a wavenumber of 3300 cm⁻¹ in a FTIR transmission spectrum.

The substantial absence of sp² hybridized CH₂ endgroups is shown
by a substantial absence of the sp² CH₂ symmetric stretching vibration at a wavenumber of 2970-2975 cm⁻¹ in a FTIR transmission spectrum; and/or
by a substantial absence of the sp² CH₂ asymmetric stretching vibration at a wavenumber of 3030-3085 cm⁻¹ in a FTIR transmission spectrum.

The substantial absence of sp³ hybridized CH₃ endgroups is shown
by a substantial absence of the sp³ CH₃ asymmetric stretching vibration at a wavenumber of 2955-2960 cm⁻¹ in a FTIR transmission spectrum; and/or
by a substantial absence of the sp³ CH₃ symmetric stretching vibration at a wavenumber of 2875 cm⁻¹ in a FTIR transmission spectrum.

For the purpose of this invention, with “substantial absence” of a specific vibration is meant that the area of the absorption band related to this specific vibration is less than 10% of the total area of the absorption bands in the wavenumber range between 2800 and 3400 cm⁻¹. Preferably, the area of the absorption band related to the specific vibration is less than 5% or even less than 1% of the total area of the absorption bands in the wavenumber range between 2800 and 3400 cm⁻¹.

For example, with a substantial absence of sp¹ CH stretching vibration at a wavenumber of 3300 cm⁻¹ is meant that the area of the absorption band with its maximum intensity at a wavenumber of 3300 cm⁻¹ is less than 10% of the total area of the absorption bands in the wavenumber range between 2800 and 3400 cm⁻¹. Preferably, the area of the absorption band with its maximum intensity at a wavenumber of 3300 cm⁻¹ is less than 5% or even less than 1% of the total area of the absorption bands in the wavenumber range between 2800 and 3400 c⁻¹.

With a substantial absence of sp² CH₂ symmetric stretching vibration at a wavenumber of 2970-2975 cm⁻¹ is meant that the area of the absorption band with its maximum intensity at a wavenumber of 2970-2975 cm⁻¹ is less than 10% and preferably less than 5% or even less than 1% of the total area of the absorption bands in the wavenumber range between 2800 and 3400 cm⁻¹.

With a substantial absence of sp² CH₂ asymmetric stretching vibration at a wavenumber of 3030-3085 cm⁻¹ is meant that the area of the absorption band with its maximum intensity at a wavenumber of 3030-3085 cm⁻¹ is less than 10% and preferably less than 5% or even less than 1% of the total area of the absorption bands in the wavenumber range between 2800 and 3400 cm⁻¹.

With a substantial absence of sp³ CH₃ asymmetric stretching vibration at a wavenumber of 2955-2960 cm⁻¹ is meant that the area of the absorption band with its maximum intensity at a wavenumber of 2955-2960 cm⁻¹ is less than 10% and preferably less than 5% or even less than 1% of the total area of the absorption bands in the wavenumber range between 2800 and 3400 cm⁻¹.

With a substantial absence of sp³ CH₃ symmetric stretching vibration at a wavenumber of 2875 cm⁻¹ is meant that the area of the absorption band with its maximum intensity at a wavenumber of 2875 cm⁻¹ is less than 10% and preferably less than 5% or even less than 1% of the total area of the absorption bands in the wavenumber range between 2800 and 3400 cm⁻¹.

Next to a substantial absence of the sp^{x} hybridized CHₓ endgroups (with x equal to 1, 2 and 3), the hydrogenated amorphous carbon coating according to the present invention is preferably further characterized by a substantial absence of the sp² hybridized CH aromatic group.

The substantial absence of the sp² hybridized CH aromatic group is clear by a substantial absence of the sp² CH aromatic stretching vibration at a wavenumber of 3050-3100 cm⁻¹ in a FTIR transmission spectrum.

With a substantial absence of sp² CH aromatic stretching vibration at a wavenumber of 3050-3100 cm⁻¹ is meant that the area of the absorption band with its maximum intensity at a wavenumber of 3050-3100 cm⁻¹ is less than 10% and preferably less than 5% or even less than 1% of the total area of the absorption bands in the wavenumber range between 2800 and 3400 cm⁻¹.

The substantial absence of sp¹ hybridized CH endgroups, of sp² hybridized CH₂ endgroups and of sp³ hybridized CH₃ endgroups implies the significant presence of sp³ hybridized CH groups and/or the significant presence of sp² hybridized CH groups.

The significant presence of sp³ hybridized CH groups is shown by a significant presence of the sp³ CH stretching vibration at a wavenumber of 2900 (±15) cm⁻¹ in a FTIR transmission spectrum.

The significant presence of sp² hybridized CH groups is shown by a significant presence of the sp² CH olefinic stretching vibration at a wavenumber of 3016 cm⁻¹ in a FTIR transmission spectrum.

With hydrogenated amorphous carbon coating is meant any amorphous coating comprising carbon and hydrogen. These coatings are often referred to as diamond-like carbon (DLC) coatings.

Preferred hydrogenated amorphous carbon coatings are coatings deposited by means of plasma enhanced chemical vapor deposition and starting from a gaseous or liquid carbon-containing precursor.

The hydrogenated amorphous carbon coating according to the present invention has preferably a sp³ content ranging between 20 and 40%, and more preferably between 20 and 30% and has a hydrogen content preferably lower than 25 at %, more preferably lower than 20 at % as for example 16 at %. The combination of this sp³ content and this hydrogen content differentiates the hydrogenated amorphous carbon coating according to the present invention from hydrogenated amorphous carbon coatings known in the art and is giving the coating according to the present invention unique characteristics.

The low hydrogen concentration is attributed to the substantial absence of the sp hybridized CH endgroups and of the sp² hybridized CH₂ endgroups and of the sp³ hybridized CH₃ endgroups.

The nanohardness of the hydrogenated carbon coating according to the present invention is preferably higher than 14 GPa, and more preferably higher than 15 GPa, for example 18 GPa or 20 GPa.

It is generally accepted in the art that the higher the sp³ C—C content of a hydrogenated amorphous carbon coating, the higher the nanohardness of the coating will be.

However, a hydrogenated amorphous carbon coating according to the present invention having a sp³ content of a hydrogenated carbon coating known in the art and having a low hydrogen content, lower than the hydrogen content of hydrogenated amorphous carbon coatings known in the art, is characterized by a high hardness.

This can be explained by the fact that the network of the hydrogenated amorphous carbon coating comprises mainly a C—C network.

A hydrogenated amorphous carbon coating according to the present invention preferably has a thickness ranging between 100 and 5000 nm and more preferably ranging between 200 and 2000 nm as for example 400 nm, 800 nm or 1200 nm.

The refractive index of a hydrogenated amorphous carbon coating according to the present invention is preferably higher than 2.2 as for example 2.4 or 2.5.

According to a second aspect of the present invention a method to deposit a hydrogenated amorphous carbon coating as described above on a substrate is provided. The method comprises the use of a remote plasma technique as for example a microwave discharge, an inductively coupled plasma or an expanding thermal plasma.

Preferably, the method comprises the use of a remote plasma characterized by a low electron temperature, typically below 0.4 eV. The method allows to deposit a hydrogenated amorphous carbon coating at a high deposition rate. The deposition rate is preferably higher than 15 nm/s as for example 20 nm/s.

One preferred method using a remote plasma comprises the use of an Expanding Thermal Plasma (ETP). The ETP deposition setup comprises one or more expanding thermal plasma sources and a low pressure deposition chamber. The ETP source preferably comprises a cascaded arc. A carrier gas (as for example argon, hydrogen, nitrogen or a mixture thereof) flows through the plasma source. This gas is ionized generating a plasma at a pressure of for example 0.5 bar. When the plasma arrives at the exit of the cascaded arc, it expands into the low pressure deposition chamber. In the deposition chamber the precursor gases necessary for the deposition are added to the plasma. The plasma mixture, which consists of the gases mentioned and the radicals, ions and electrons originating thereof, is transported subsonically towards the substrate.

The ETP deposition technique according to the present invention allows depositing hydrogenated amorphous carbon coatings with a high deposition rate.

The deposition rate of a hydrogenated amorphous carbon coating deposited by the ETP deposition technique is preferably higher than 15 nm/s and more preferably higher than 20 nm/s as for example 40 nm/s or 60 nm/s.

A preferred method to deposit a hydrogenated amorphous carbon coating on a substrate uses a remote plasma is provided whereby the chemistry within the plasma is tailored in such a way that the coating is deposited on the substrate with a deposition rate of at least 15 nm/s.

More preferably, the deposition rate is at least 20 nm/s as for example 40 nm/s or 60 nm/s.

The ratio of the carrier gas flow emanating from the ETP source to the flow of introduced carbon containing gas is preferably lower than 10, for example 5, 2 or 1.

Examples of carbon-containing gas comprise methane, ethane, ethylene, acetylene, propane, butane, benzene and toluene.

The ratio of the carrier gas flow emanating from the ETP source to the flow of introduced carbon containing precursor gas has a significant influence on the properties of the hydrogenated amorphous carbon coating.

### BRIEF DESCRIPTION OF THE DRAWINGS

The invention will now be described into more detail with reference to the accompanying drawings wherein

FIG. 1 is an illustration of FTIR transmission spectra of a hydrogenated amorphous carbon coating according to the present invention and of a hydrogenated amorphous carbon coating known in the art;

FIG. 2 is an illustration of the fitted FTIR transmission spectra of FIG. 1; and

FIG. 3 is an illustration of the first derivative of the FTIR transmission spectra of FIG. 1.

### DESCRIPTION OF THE PREFERRED EMBODIMENTS OF THE INVENTION

A number of different hydrogenated amorphous carbon coatings are deposited under different conditions.

Coating 1 comprises a hydrogenated amorphous carbon coating according to the present invention. Coating 1 is deposited using an expanding thermal plasma at a deposition rate of 24 nm/s.

Coating 2 comprises a hydrogenated amorphous carbon coating deposited as described in J. Appl. Phys. 80 (10), 5986-5995, 1996.

Coating 3 comprises a hydrogenated amorphous carbon coating deposited by plasma enhanced chemical vapour deposition.

Some properties (hardness, sp³ content and hydrogen content, refractive index) are compared in Table 1. The sp³ content and the hydrogen content of the three coatings are determined by Raman Spectroscopy. The refractive index of the three coatings is determined by Spectroscopic Ellipsometry at a wavelength of 632 nm.

TABLE 1

| | Coating 1 | Coating 2 | Coating 3 |
|---|---|---|---|
| deposition rate | 24 | 3 | 0.25 |
| (nm/s) | | | |
| Hardness | 17 | 12.5 | 21 |
| (GPa) | | | |
| sp³ content | 30 | 40 | 40 |
| (%) | | | |
| hydrogen content | 22 | 33 | 27 |
| (at %) | | | |
| Refractive index | 2.5 | 2.2 | 2.3 |

From Table 1 it can be concluded that coating 1, although having a lower sp^{a} content compared to coating 2 and coating 3, is characterized by a rather low hydrogen content and a high hardness.

Fourier Transform InfraRed (FTIR) spectroscopy is used for the qualitative characterization of the hybridization and bonding configuration of the different types of hydrogenated amorphous carbon coatings (Coatings 1 to 3).

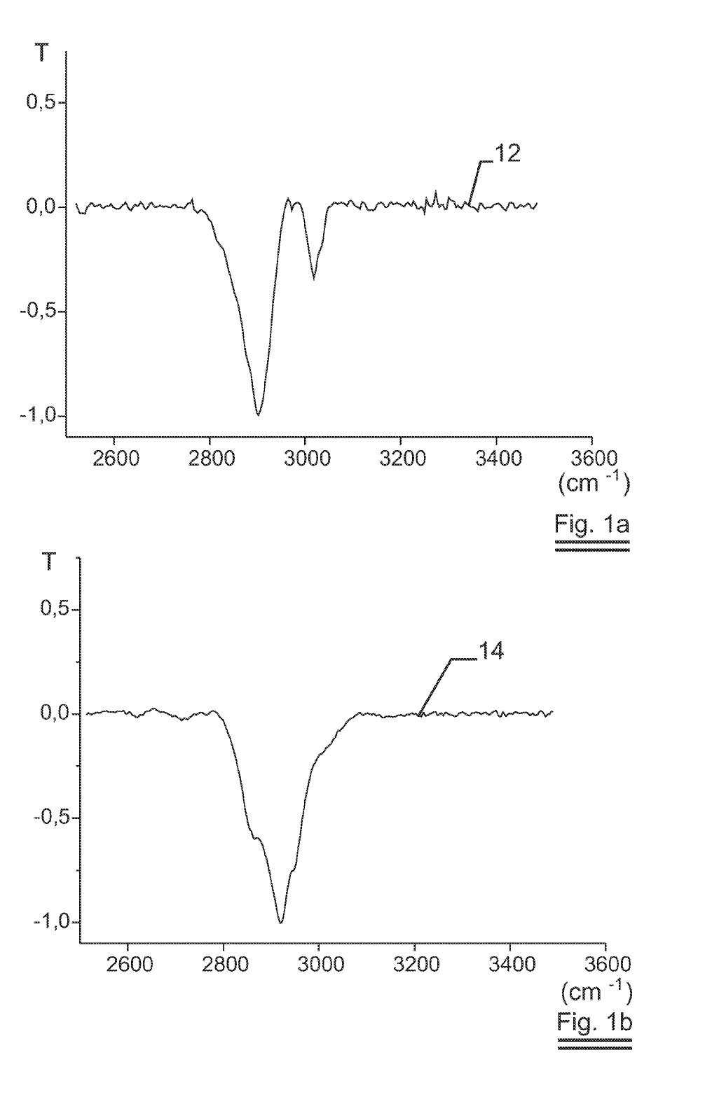

In FIG. 1 the transmission spectra obtained by FTIR spectrosopy of a hydrogenated amorphous carbon coating according to the present invention (Coating 1) and of a hydrogenated amorphous carbon coating of the prior art (Coating 2) are visualized. The FTIR transmission spectrum of the hydrogenated amorphous carbon coating according to the present invention is given by spectrum 12 in FIG. 1a. The FTIR transmission spectrum of the hydrogenated amorphous carbon coating of the prior art is given by spectrum 14 in FIG. 1 b. In the X-axis the wavenumbers are given, the Y-axis shows the transmission.

Spectrum 12 is clearly different from spectrum 14. Spectrum 12 shows two peaks separated by a valley, whereas spectrum 14 shows one broad peak between 2800 and 3400 cm⁻¹.

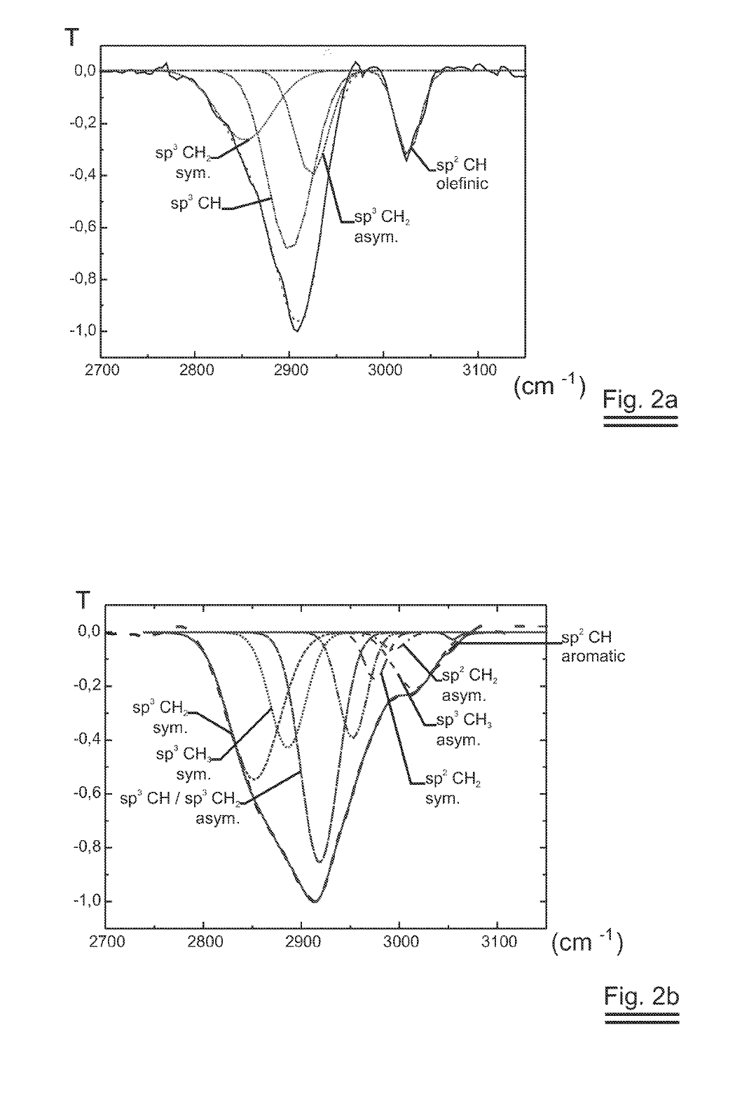

The FTIR transmission spectra 12 and 14 have been fitted in the wavenumber range from 2800 cm⁻¹ to 3400 cm⁻¹. The fitted FTIR transmission spectrum 12 is given in FIG. 2a; the fitted FTIR transmission spectrum 14 is given in FIG. 2b.

To obtain the fitted FTIR transmission spectrum, first the interference background is determined by measuring the FTIR transmission spectrum of a blank sample. After subtraction of the interference background, the individual absorption peaks representing the specific stretching vibrations are determined. In the fit procedure each absorption peak is represented by a Gaussian function. For the fit procedure the peak positions are kept fixed. The parameters that vary are thus the peak height and the peak width.

The stretching vibrations and corresponding bonding types used are given in Table 2. These vibrations correspond with vibrations given in

J. Appl. Phys., Vol. 84, No. 7, p. 3836-3847, 1998, Table I and Table II and in Solid State Comm., Vol. 48, No. 2, p. 105-108, 1983, Table II.

TABLE 2

| Stretching vibration | | |
|---|---|---|
| [cm⁻¹] | | Bonding type |
| 2850 | cm⁻¹ | sp³ CH₂ symmetric stretching |
| 2875 | cm⁻¹ | sp³ CH₃ symmetric stretching |
| 2900 | cm⁻¹ | sp³ CH stretching |
| 2924 | cm⁻¹ | sp³ CH₂ asymmetric stretching |
| 2955-2960 | cm⁻¹ | sp³ CH₃ asymmetric stretching |
| 2970-2975 | cm⁻¹ | sp² CH₂ symmetric stretching |
| 3016 | cm⁻¹ | sp² CH olefinic stretching |
| 3030-3085 | cm⁻¹ | sp² CH₂ asymmetric stretching |
| 3050-3100 | cm⁻¹ | sp² CH aromatic stretching |
| 3300 | cm⁻¹ | sp¹ CH stretching |

From FIG. 1 and FIG. 2 it is clear that the FTIR transmission spectrum of a hydrogenated amorphous carbon according to the present invention is different from the FTIR transmission spectrum of a hydrogenated amorphous carbon coating of the prior art in the wavenumber range between 2800 cm⁻¹ and 3400 cm⁻¹.

This difference is due to the substantial absence of sp¹ hybridized CH endgroups, to the substantial absence of sp² hybridized CH₂ endgroups and to the substantial absence of sp³ hybridized CH₃ endgroups in the coating according to the present invention.

The substantial absence of sp¹ hybridized CH endgroups is shown by a substantial absence of the sp¹ CH stretching vibration at a wavenumber of 3300 cm⁻¹.

The substantial absence of sp² hybridized CH₂ endgroups is shown by a substantial absence of the sp² CH₂ symmetric stretching vibration at a wavenumber of 2970-2975 cm⁻¹, and/or by a substantial absence of the sp² CH₂ asymmetric stretching vibration at a wavenumber of 3030-3085 cm⁻¹ in a FTIR transmission spectrum.

The substantial absence of sp³ hybridized CH₃ endgroups is shown by a substantial absence of the sp³ CH₃ asymmetric stretching vibration at a wavenumber of 2955-2960 cm⁻¹ and/or by a substantial absence of the sp³ CH₃ symmetric stretching vibration at a wavenumber of 2875 cm⁻¹ in a FTIR transmission spectrum.

Furthermore, the coating according to the present invention is characterized by the presence of sp³ hybridized CH groups and by the presence of sp² hybridized CH groups shown by the presence of the sp³ CH stretching vibration at a wavenumber of 2900 (±15) cm⁻¹ in a FTIR transmission spectrum and by the presence of the sp² CH olefinic stretching vibration at a wavenumber of 3016 cm⁻¹ in a FTIR transmission spectrum.

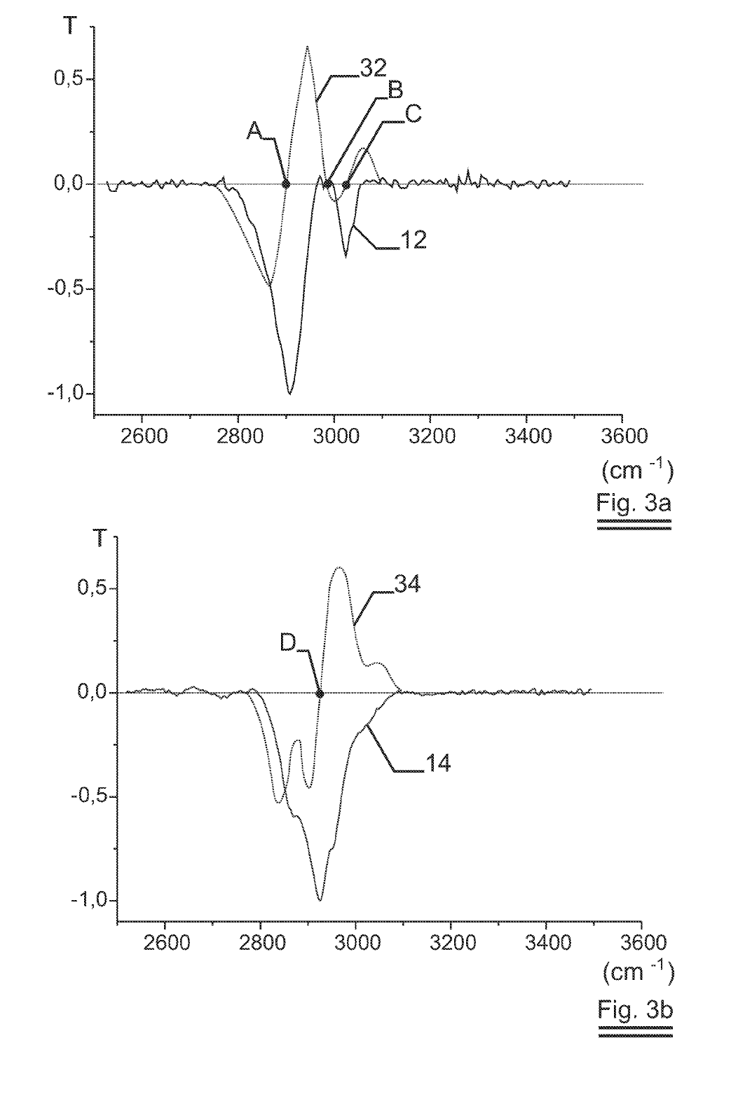

FIG. 3 shows the first derivative of the FTIR transmission spectra given in FIG. 1. The first derivative of the FTIR transmission spectrum of a coating according to the present invention is given by spectrum 32 in FIG. 3a. The first derivative of the FTIR transmission spectrum of a coating of the prior art is given by spectrum 34 in FIG. 3b. In the X-axis the wavenumbers are given, the Y-axis shows the transmission.

Spectrum 32 of FIG. 3a has three zero axis crossings in the wavenumber range between 2850 and 3050 cm⁻¹. The zero axis crossings are indicated in FIG. 3a with A, B and C.

The first zero axis crossing A is corresponding with the maximum absolute intensity of the first peak in the FTIR transmission spectrum.

The second zero axis crossing B is corresponding with the second minimum of the absolute intensity in the FTIR transmission spectrum, i.e. with the peak valley. The third zero axis crossing C is corresponding with the maximum absolute intensity of the second peak in the FTIR transmission spectrum.

Spectrum 34 of FIG. 3b has one zero axis crossings in the wavenumber range between 2850 and 3150 cm⁻¹. The zero axis crossing is indicated in FIG. 3b with D.

The zero axis crossing D is corresponding with the maximum absolute intensity of the peak in the FTIR transmission spectrum.

1. A hydrogenated amorphous carbon coating characterized by a substantial absence of the sp^{x} hybridized CHₓ endgroups whereby x is equal to 1, 2 or 3.

2. A hydrogenated amorphous carbon coating according to claim 1, wherein the first derivative of the Fourier Transform InfraRed (FTIR) transmission spectrum of the hydrogenated amorphous carbon coating has in the wavenumber range between 2850 and 3050 cm⁻¹ at least three zero axis crossings, a zero axis crossing being the intersection of the first derivative with the axis Y=0.

3. A hydrogenated amorphous carbon coating according to claim 1, characterized by a substantial absence of sp^{x} hybridized CHₓ endgroups wherein x is equal to 1, 2 or 3, whereby wherein said substantial absence of sp^{x} hybridized CHₓ endgroups is defined as a substantial absence of the sp^{x} CHₓ stretching vibration or vibrations in a Fourier Transform InfraRed transmission spectrum, a substantial absence of a sp^{x} CHₓ stretching vibration is defined as a specific sp^{x} CHₓ stretching vibration having an area which is less than 10% of the total area of the absorption bands in the wavenumber range between 2800 and 3400 cm⁻¹.

4. A hydrogenated amorphous carbon coating according to claim 1, wherein said substantial absence of sp¹ hybridized CH endgroups is shown by a substantial absence of the sp¹ CH stretching vibration at a wavenumber of 3300 cm⁻¹ in a Fourier Transform InfraRed transmission spectrum.

5. A hydrogenated amorphous carbon coating according to claim 1,wherein 2, whereby said substantial absence of sp² hybridized CH₂ endgroups is shown by a substantial absence of the sp² CH₂ symmetric stretching vibration at a wavenumber of 2970-2975 cm⁻¹ in a Fourier Transform InfraRed transmission spectrum; and/or by a substantial absence of the sp² CH₂ asymmetric stretching vibration at a wavenumber of 3030-3085 cm⁻¹ in a Fourier Transform InfraRed transmission spectrum.

6. A hydrogenated amorphous carbon coating according to claim 1, wherein said substantial absence of sp³ hybridized CH₃ endgroups is shown by a substantial absence of the sp³ CH₃ asymmetric stretching vibration at a wavenumber of 2955-2960 cm⁻¹ in a Fourier Transform InfrarRed transmission spectrum; and/or by a substantial absence of the sp³ CH₃ symmetric stretching vibration at a wavenumber of 2875 cm⁻¹ in a Fourier Transform InfraRed transmission spectrum.

7. A hydrogenated amorphous carbon coating according to claim 1, wherein said coating is further characterized by a substantial absence of the sp² hybridized CH aromatic group.

8. A hydrogenated amorphous carbon coating according to claim 7, wherein said substantial absence of the sp² hybridized CH aromatic group is shown by a substantial absence of the sp² CH aromatic stretching vibration at a wavenumber of 3050-3100 cm⁻¹ in a Fourier Transform InfraRed transmission spectrum.

9. A hydrogenated amorphous carbon coating according to claim 1, wherein said coating has a sp³ content ranging between 20 and 40% and a hydrogen content lower than 25%.

10. A hydrogenated amorphous carbon coating according to claim 1, wherein said coating has a nanohardness higher than 14 GPa.

11. A hydrogenated amorphous carbon coating according to claim 1, wherein said coating has a refractive index higher than 2.2.

12. A hydrogenated amorphous carbon coating according to claim 1, wherein a diamond-like carbon coating is deposited at a deposition rate higher than 15 nm/s.

13. A hydrogenated amorphous carbon coating according to claim 1, wherein a diamond-like carbon coating is deposited by using a remote plasma technique.

14. A method to deposit a hydrogenated amorphous carbon coating as defined in claim 1 on a substrate, wherein said method comprises the use of a remote plasma technique.

15. A method according to claim 14, wherein said remote plasma has an electron temperature lower than 0.4 eV.

16. A method according to claim 14, wherein said remote plasma comprises an expanding thermal plasma.

17. A method according to claim 14 using a remote plasma whereby the chemistry within said plasma is tailored in such a way that said coating is deposited on said substrate with a deposition rate higher than 15 nm/s.

18. A method according to claim 14, wherein the ratio of inert gas ion flow to the flow of introduced carbon containing precursor gas is lower than 10.
